(19) United States
(12) Patent Application Publication
(10) Pub. No.: US 2013/0035636 A1
(43) Pub. Date: Feb. 7, 2013

(54) Delivery and Deployment Catheter for an Implantable Medical Device

(71) Applicants: Rudy Beasley, Rohnert Park, CA (US); William Chang, Santa Rosa, CA (US); Sina Som, Santa Rosa, CA (US); Don Tran, Novato, CA (US); James Calvin Allan, Santa Rosa, CA (US)

(72) Inventors: Rudy Beasley, Rohnert Park, CA (US); William Chang, Santa Rosa, CA (US); Sina Som, Santa Rosa, CA (US); Don Tran, Novato, CA (US); James Calvin Allan, Santa Rosa, CA (US)

(73) Assignee: Medtronic Vascular, Inc., Santa Rosa, CA (US)

(21) Appl. No.: 13/197,072

(22) Filed: Aug. 3, 2011

(51) Int. Cl. A61M 25/092

(52) U.S. Cl. 604/95.01

(57) ABSTRACT

A delivery catheter and method for delivering and deploying an implantable medical device include a mechanical latch by which the device can remain firmly attached at a single point of connection to the delivery catheter during deployment of the device. The delivery catheter provides simple yet firm control of the release mechanism to enable the clinician to confirm the accuracy of the deployment before releasing the tether or, if the placement of the device should be corrected, the tether can be maintained while the device is recaptured and repositioned or removed from the patient.

### FIELD OF THE INVENTION

The invention relates to delivery and deployment systems for implantable medical devices.

### BACKGROUND

Various implantable medical devices have been clinically implanted or proposed for therapeutically treating or monitoring one or more physiological conditions of a patient. Such devices may be adapted to monitor or treat conditions or functions relating to heart, blood vessels, muscle, nerve, brain, stomach, endocrine organs or other organs and their related functions. Advances in design and manufacture of miniaturized medical devices have resulted in devices capable of therapeutic as well as diagnostic functions such as pacemakers, cardioverters, defibrillators, biochemical sensors, pressure sensors, various endovascular devices and the like. Such devices may have electronic functions and may be associated with electrical leads or may be wireless, with the ability to transmit data electronically either to another device implanted in the patient or to another device located externally of the patient, or both. Such devices also may include purely mechanical functions

Although implantation of some devices requires a surgical procedure (e.g., pacemakers, defibrillators, etc.) other devices may be small enough to be delivered and placed at an intended deployment site in a relatively noninvasive manner, such as by a delivery catheter introduced percutaneously. Delivery also may be accomplished by advancing a catheter intravascularly through an exposed vessel during a surgical procedure. Depending on the nature, function and intended deployment site of the device, the manner in which the device is fixed in place and oriented in the body may affect the operation and accuracy of the device. Consequently, the accuracy of means by which the device is fixed in place in the body can be a significant factor in the performance and utility of the device.

For those devices intended for placement by a delivery catheter, the ability to reliably place the medical device in the desired position and orientation may present some difficulty, depending on variables such as the intended placement site, the patient's anatomy and the device design. During advancement of the catheter, its positioning and deployment of the device is monitored fluoroscopically while the distal end of the catheter is navigated and manipulated from its proximal end. Typically, the device is carried at the distal end of the catheter in a low profile, radially compressed configuration to facilitate navigation of the catheter. When the catheter has been navigated to the target site and is operated to release and deploy the device, the device expands or is expanded to its larger profile into engagement with the vessel.

It is not uncommon, however, for self-expanding devices, when released from the delivery catheter, to shift position or orientation from that intended by the clinician. This may result from a number of causes as will be recognized by those familiar with the art. For example, in the case of a stent or spring-like support for a sensor or the like, the springy nature of the device may cause it to jump out of position before it has engaged the vessel wall sufficiently to assume a secure placement in the vessel. In such circumstances it would be desirable to recapture the device in the catheter to enable it to be repositioned and redeployed. Delivery catheters have been described to permit recapture of the medical device for that purpose.

It is among the general objects of the invention to provide an improved delivery catheter adapted to engage a medical device in a manner that allows for a controlled release of the device and allows the device to be recaptured, repositioned and redeployed at the intended site.

### SUMMARY OF THE INVENTION

In accordance with the invention the delivery catheter is arranged to engage and contain the medical device within the distal end of a sheath in a low profile delivery configuration. The catheter also includes a retention mechanism that releasably engages the medical device at a single point of connection by which the medical device may be securely held during and after its expansion at the deployment site. The retention mechanism enables the clinician to release the connection to the device only after it is determined that the location and orientation of the deployed device is satisfactory. If not, it enables the clinician to maintain the position of the medical device while recapturing the device in the sheath either for repositioning or removal from the patient.

The delivery catheter is suited particularly for use with implantable devices that have a loop at the proximal end to which the retention mechanism may attach at a single connection point. The loop may be formed in one unitary piece with the medical device or may be separately attached to form an integrated unit. For example the device may be of the types described in U.S. patent applications No. 13/109,409 filed May 17, 2011 and No. 13/090,854 filed Apr. 20, 2011 in which the device has a sensor or the like attached to a fixation member that has a loop extending proximally from the proximal end of the fixation member. The disclosures of those applications are incorporated by reference herein, in their entireties. As described in those applications the implantable device preferably has a configuration at its proximal region that facilitates its progressive radial contraction as the sheath is progressively advanced distally over the device to effect recapture.

The delivery device has an elongate multi-component shaft that includes an outer sheath, an inner sheath telescopically contained in the outer sheath and an elongate inner core extending through the inner sheath, all of which are moveable longitudinally relative to each other. The retention mechanism includes a retainer mounted to the distal region of the inner sheath. The retainer is configured to normally engage the loop at the proximal end of the implantable device to secure the position of the device with respect to the inner sheath until the retainer is triggered to release the loop of the implantable device. The retainer is maintained in latched engagement with the loop as the outer sheath is retracted proximally to enable expansion of the device from its low profile configuration to its deployed configuration in engagement with the wall of the vessel. The retainer thus serves to maintain a tether to the device during deployment to prevent the device from shifting position during the deployment. While the tether is maintained the device can be recaptured by advancing the sheath distally. Only after the clinician is satisfied that the device has been placed properly in the vessel is the retainer triggered to release its connection to the device. The release is triggered in response to longitudinal movement of the inner core relative to the inner sheath that causes a cam on the core to engage the retainer in a manner that releases its connection to the loop of the device.

### THE DRAWINGS

The various objects and advantages of the invention will be appreciated further from the following detailed description with reference to the accompanying drawings in which:

FIG. 1 is a fragmented cross-sectional illustration of the delivery catheter loaded with an implantable device in readiness for use and with the distal region shown in enlarged detail and with the proximal, handle end of the device shown in reduced scale;

FIG. 2 is a diagrammatic illustration of the distal region of the catheter in which the deployment sequence has been initiated by advancing the inner core distally;

FIG. 3 is a diagrammatic illustration of the system in which the outer sheath is partially withdrawn to initiate the manner in which the implantable device is partially deployed or recaptured;

FIG. 4 shows the system with the outer sheath having been fully withdrawn and with the implantable device having been expanded as it would be into contact with the wall of the target vessel (not shown);

FIG. 5 shows the system after the core has been retracted proximally to trigger the retainer and release the device;

FIG. 6 is a longitudinal sectional illustration of the distal end of the system, also when the core has been fully retracted as in FIG. 5 with the retainer actuated to release the medical device;

FIG. 7 is a plan view of the distal end of the inner sheath and retainer showing the recessed grooves that receive the retainer and proximal end of the device proximal loop.

### DESCRIPTION OF THE ILLUSTRATIVE EMBODIMENT

Specific embodiments of the present invention are now described with reference to the figures, wherein like reference numbers indicate identical or functionally similar elements. The terms “distal” and “proximal” are used in the following description with respect to a position or direction relative to the treating clinician. “Distal” or “distally” are a position distant from or in a direction away from the clinician. “Proximal” and “proximally” are a position near or in a direction toward the clinician.

The delivery catheter is adapted to be used with an implantable device having a loop-defining structure at its proximal end by which it may be tethered to the catheter. Although the invention is described as being used with devices such as those described in U.S. patent application Ser. Nos. 13/109,409 and 13/090,854, it also may be used to deliver other types of devices such as stents that have at least one loop-defining apex or crown or other member that includes at least a U-shaped portion. As described in those applications the implantable device includes an expandable fixation member to which a sensor may be mounted, in which the fixation member has a proximally extending loop by which it can be engaged by a tether at a single location.

As shown in the drawings, the delivery system or catheter comprises an elongate, flexible multi-component shaft 10 having proximal and distal ends 12, 14, respectively. A handle 16 is attached at the proximal end of shaft 10 and the catheter distal end terminates in a generally tapered or conical tip 18. The multi-component shaft 10 includes an elongate outer sheath 20 that contains an inner sheath 22 that, in turn, contains a tubular core member 24, longer than either of the sheaths. Tip 18 is attached to the distal end of core 24. Each of outer sheath 20, inner sheath 22 and core member 24 is moveable longitudinally with respect to the others.

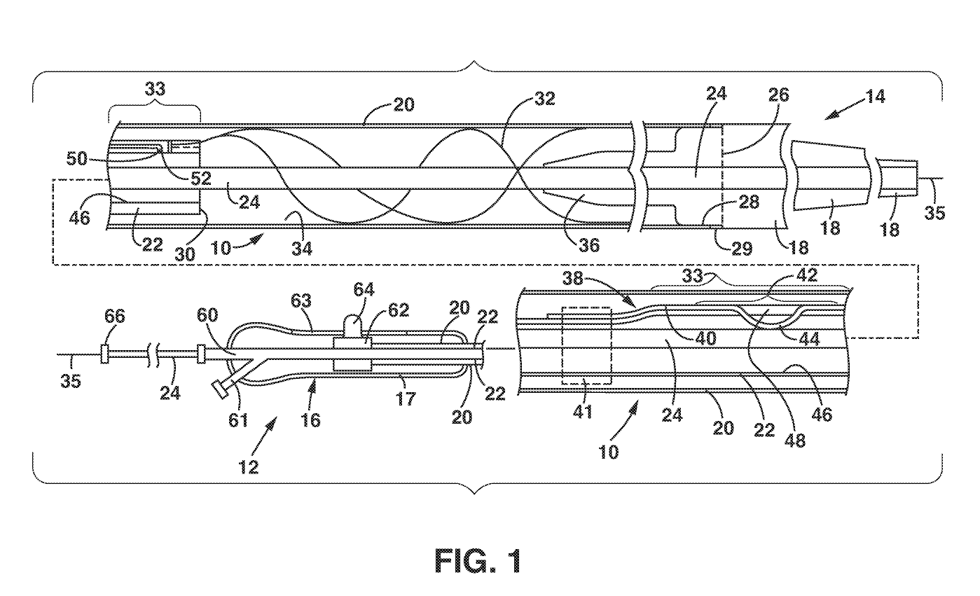

Outer sheath 20 has a lumen 34 extending therethrough and a distal end terminating in a distal opening 26 that mates with a proximally facing shoulder 28 formed on distal tip 18 to form a smooth transition of the outer surfaces of sheath 20 and tip 18 when the two are mated in the delivery configuration of FIG. 1. By way of example, the outer sheath may have an inner diameter of about 0.168 inch and an outer diameter of about 0.184 inch. It may be extruded from polyether block amide copolymer (PEBA) having 55 shore D durometer or, alternatively, may be formed as a reinforced tube having an inner polytetrafluoroethylene (PTFE) liner, an intermediate reinforcing layer of braided stainless steel and an outer jacket of 55D durometer PEBA. The distal end of outer sheath 20 preferably include a radiopaque ring 29 that may be formed by incorporating barium sulfate or other suitable radiopaque material such as tungsten into or at the end of the sheath 20.

Inner sheath 22 may have inner and outer diameters of about 0.050 inch and 0.070 inch, respectively, and may be formed from 70D durometer PEBA sold under the trademark PEBAX 7033. When the catheter is in its delivery configuration the implantable device is contained within the distal end of outer sheath 20 and the distal end 30 of inner sheath 22 is located proximally of the implantable device 32 except for where the proximal loop or tip of the device is in engagement with the inner sheath. The distal region 33 of inner sheath 22 is enlarged in diameter and wall thickness. For example, in the illustrative embodiment region 33 may have an inner diameter of about 0.100 inch and an outer diameter of about 0.150 inch. The enlarged diameter region may be of the order of about one centimeter long. It may be formed as a separate piece that is joined to the distal end of the smaller diameter proximal section.

Tubular core 24 has a lumen 31 receptive to a medical guidewire as suggested at 35 in the drawings. The tubular core may be formed from polyimide reinforced with stainless steel braiding and may have an outer diameter of about 0.031 inch and an inner diameter of about 0.021 inch for use with a 0.018 inch diameter guidewire. For a guidewire having a diameter of 0.035 inch, core 24 may have an inner diameter of about 0.038 inch and an outer diameter of about 0.046 inch. Distal tip 18 is attached to the distal end of core 24 and may be formed from 35D durometer PEBA or a blend of 40D durometer PEBA and barium sulfate. Tip 18 has a lumen 31 that is a continuation of core lumen 34. In the illustrative example the outer diameter of distal tip 18 may be 0.184 inch to match the outer diameter of outer sheath 20. As described in further detail below, an actuating cam 36 is mounted to core 24 in a position to enable it to trigger release of the implantable device from the retaining mechanism. Cam 36 may be formed as an integral part of distal tip 18 or may be formed separately and attached to core 24.

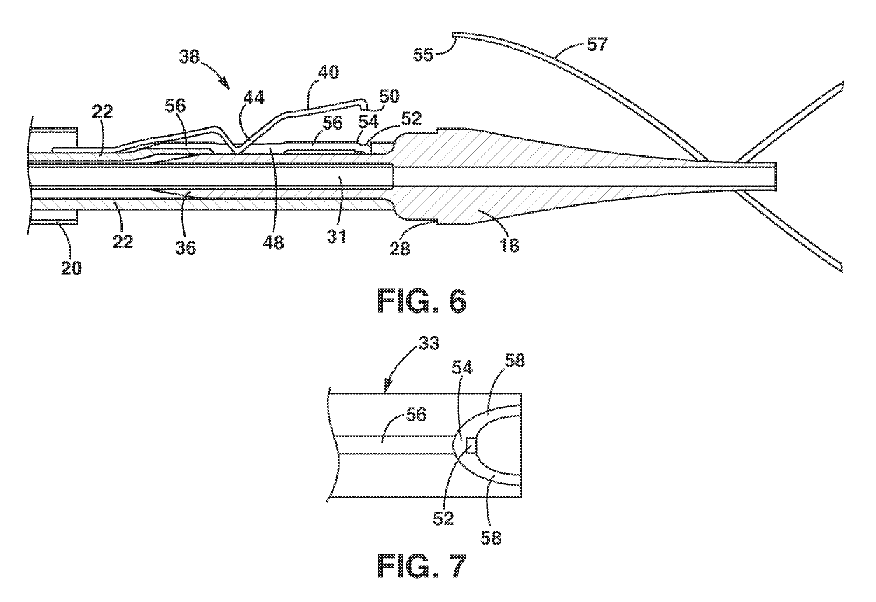

The delivery catheter includes a retention mechanism, indicated generally at 38, by which the implantable device 32 can be tethered to the catheter until the clinician desires to release it from the delivery catheter (see also FIG. 6). The mechanism 36 includes a retainer 40 that serves as a latching element and may be in the form of an elongate resilient strip that may be fabricated, for example, from stainless steel, nickel-titanium (nitinol), cobalt-chromium-nickel superalloy sold under the trademark ELGILOY, or other suitable material. Retainer 40 is mounted at its proximal end to the outer surface of the inner sheath 22 and may be secured by shrink tubing 41 firmly constricted about its proximal end and the inner sheath. An intermediate portion 42 of retainer 40 is shaped, e.g. V-shaped, to define a follower 44 that extends radially into the lumen 46 of the inner sheath through a window 48 formed in the wall of inner sheath 22. The distal portion of the retainer extends along the exterior of the sheath 22. The end of retainer 40 is bent downwardly to define a distal finger 50 that is received in a socket or hole 52 formed in inner sheath 22. The spring force or resilience of retainer 40 normally biases it against the exterior surface of the inner sheath to engage finger 50 in socket 52 with follower 44 extending into the lumen of the inner sheath through window 48. The arrangement enables a proximal loop portion of the implantable device to be entrapped by spring loaded retainer 40 in a recess 54 in the outer surface of the region 33 immediately proximal of the socket. Implantable device 32 may be released by drawing core 24 proximally relative to inner sheath 22 to cause cam 36 to engage follower 44 and urge the follower radially outwardly, thus elastically bending retainer 40 and urging finger 50 out of socket 52 and enabling release of the implantable device. By way of illustrative example, retainer 40 may be of the order of about 0.012 inch wide, 0.007 inch thick and about ten millimeters in length.

As shown in more detail in FIG. 7 grooves may be formed on the external surface of the distal portion of the region 33 of the inner sheath to enable retainer 40 as well as the proximal end 55 of the loop or apex 57 of the implantable device 32 to be recessed within the thickness of the wall of distal region 33 of inner sheath 22. A U-shaped groove 54 may be provided in the end of distal region 33 to receive proximal end 55 of the proximal loop 57 of the implantable device. Groove 54 has two legs 58 extending distally from an apex located immediately proximal to the socket 52. Retainer 40, when in its latched configuration, retains the proximal end of the loop 57 in the U-shaped groove 54 and prevents the implantable device from shifting proximally or distally during retraction of the outer sheath. One longitudinally extending groove 56 extends proximally from socket 52 through window 48 to the proximal end of region 33. Groove 56 enables the portion of retainer 40 that extends along distal region 33 to be recessed until the time that the implantable device is to be released.

Operation of the catheter may be controlled from the handle 16 at the system proximal end 12. The proximal end of inner sheath 22 extends into the handle and is attached, as by bonding, at its proximal end to a two-port luer fitting 60 that is securely mounted in the proximal end of the housing 17 that forms the handle. The proximal end of outer sheath 20 extends into the handle and is attached to a slide 62. Slide 62 is movably mounted about and guided along inner sheath 22 within handle 16. Slide 62 is operated by a thumb button 64 that extends out of the side of housing 17 through a longitudinal slot 63. Moving button 64 proximally or distally, as described in more detail below, controls the relative longitudinal positions of inner and outer sheaths 20, 22. The respective lengths of the sheaths and slide control enable a range of movement in which the distal end of outer sheath 20 can be moved between an extended distal position and a retracted proximal position that are distally beyond the distal end of inner sheath 22 and proximally of the retention mechanism, respectively.

Inner core 24 extends through inner sheath 22 and through the straight-through port of two-port luer fitting 60. Side port 61 of the luer fitting enables fluid communication with the patient's vessel through inner sheath 22. A cap 66 is removably attached to the proximal protruding end of core 24 and may be used to manipulate the longitudinal position of the core relative to the other shaft components.

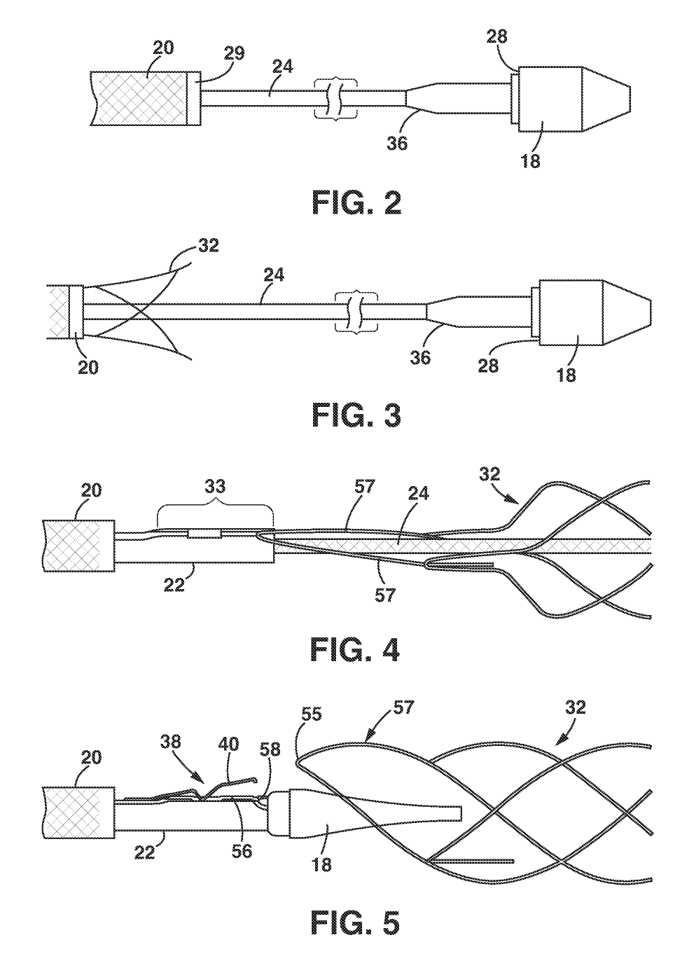

FIGS. 2-6 illustrate, sequentially, the manner in which the delivery system is operated to deliver, deploy and release or reposition or remove the medical device if the procedure is aborted. FIG. 1 shows the system with the medical device loaded into the distal end of outer sheath 20, with its proximal loop 57 captured by retainer 40 and with core 24 positioned with shoulder 28 of tip 18 nested against distal opening 26 of outer sheath 20. Cam 36 is mounted on core 24 so that in this configuration it will be located distally of retention mechanism 38. In this configuration slide 62 and thumb button 64 are located midway along slot 63 in handle 16 and may be considered to be in a neutral position.

The device may be loaded over guidewire 35 and advanced into the patient's entry vessel, such as the femoral artery, and then manipulated and navigated through the patient's vasculature until the medical device is positioned at the target site where it is to be deployed. Once positioned, the deployment procedure may be initiated by extending core 24 distally so that distal tip 18 will be clear of the deployment operation (FIG. 2). Outer sheath 20 then is withdrawn by sliding thumb button 64 proximally, thus progressively exposing medical device 32 (FIG. 3). If device 32 is self-expanding, it may begin to expand from its low profile, constrained configuration as the sheath withdrawal progresses. The device is prevented from shifting its position or unexpectedly springing distally because it is held in longitudinal position by the cooperation of retainer 40 and recess groove 54 at the end of inner sheath 22. The arrangement also prevents device 32 from being dragged proximally in response to the retraction of outer sheath 20. Withdrawal of outer sheath 20 is continued until the medical device has been fully uncovered and has self-expanded or been expanded radially against the vessel wall (not shown) (FIG. 4).

The clinician then may observe the position and orientation of medical device 32, e.g. fluoroscopically, to determine if it has been placed properly. If so, it can be released from retention mechanism 38 and the delivery system then may be withdrawn from the patient. With outer sheath 20 maintained in its retracted position, device 32 is released by retracting core 24 proximally within inner sheath 22. As cam 36 on core 24 engages follower 44 of retainer 40 it will resiliently bend the retainer radially outwardly to disengage finger 50 from its socket 52 and release loop 57 of medical device 32. With the device so deployed, the multi-component shaft 10 then can be removed.

In the event that the position or placement of medical device 32 is unsatisfactory, the clinician can recapture the medical device by maintaining retainer mechanism 38 in engagement with loop 57 of the device and advancing outer sheath 20 distally over the device by operating thumb button 64. When the device has been recaptured in the outer sheath, core 24 is retracted proximally to engage shoulder 28 of tip 18 to abut distal opening 26 of outer sheath 20. The delivery system then can be manipulated to reposition implant device 32 as desired, followed by the deployment procedure described above. Alternatively, the recaptured device may be removed from the patient together with the delivery catheter.

Thus, the invention provides a simple, easily operated system for deploying a medical device in a body lumen that reduces the risk of the device shifting out of position during deployment by maintaining a firm, mechanical tether at a single point of connection to the device until it is confirmed that it has been deployed as desired. It should be understood, however, that the foregoing description of the invention is intended merely to be illustrative and that other modifications, embodiments and equivalents that incorporate the principles of the invention may be apparent to those skilled in the art.

We claim:

1. A delivery catheter for delivering and deploying an expandable medical device having a loop at its proximal end, the catheter having proximal and distal ends and comprising:
an elongate outer sheath having a distal opening, the distal portion of the outer sheath being receptive to the expandable medical device in a radially contracted configuration;
an elongate inner sheath extending through the outer sheath, the outer sheath being movable longitudinally over the inner sheath between an extended position in which the distal end of the outer sheath extends distally beyond the distal end of the inner sheath and a retracted position in which the distal end of the outer sheath is disposed proximally of the distal end of the inner sheath;
a retainer mounted to the distal region of the inner sheath, the retainer having a latching element that is movable between a latched position in which it securely engages the loop of the device at a single location and an unlatched position in which the loop is released from the retainer; and
a control member disposed within the inner sheath and operatively associated with the retainer to operate the retainer between its latched and unlatched positions.

2. The delivery catheter as defined in claim 1 wherein the retainer has a follower extending radially into the inner sheath, the control member being longitudinally movable within the inner sheath and engageable with the follower to move the retainer from its latched to its unlatched position.

3. The delivery catheter as defined in claim 2 wherein the retainer further comprises:
an elongate, flexible, resilient strip having proximal and distal ends, the strip being attached at its proximal end to the distal region of the inner sheath with the distal end of the strip being movable radially toward and away from the inner sheath, the strip extending substantially parallel to the inner sheath when the strip is in its latched position;
the distal end of the strip having a radially inwardly extending finger;
the distal region of the inner sheath having a socket aligned with and receptive to the finger when the strip is in its latched position.

4. The delivery catheter as defined in claim 3 further comprising:
The inner sheath having a window formed at a location between the ends of and in alignment with the strip;
the follower being located at an intermediate portion of the strip, the follower projecting radially inwardly through the window into the inner sheath where it may be operatively engaged by the control member.

5. The delivery catheter as defined in claim 4 wherein the follower comprises an intermediate segment of the strip being formed in a V-shape.

6. The delivery catheter as defined in claim 2 wherein the control member comprises an elongate core extending through and movable longitudinally through the inner sheath, and a cam mounted to and movable with the core, the core being movable distally of an initial position distal of the follower and proximally of the initial position to an extent to enable the cam to engage the follower.

7. The delivery catheter as defined in claim 3 wherein the control member comprises an elongate core extending through and movable longitudinally through the inner sheath, and a cam mounted to and movable with the core, the core being movable distally of an initial position distal of the follower and proximally of the initial position to an extent to enable the cam to engage the follower.

8. The delivery catheter as defined in claim 6 wherein the core extends proximally and distally beyond the proximal and distal ends, respectively, of the outer sheath and is movable independently of each sheath; and
a distal tip attached to the distal end of the core, the tip having a proximally facing surface adapted to mate smoothly with the distal end of the outer sheath.

9. The delivery catheter as defined in claim 8 wherein the distal end of the outer sheath and the distal tip are radiopaque

10. The delivery catheter as defined in claim 8 wherein the cam is formed integrally with the tip and extends proximally a distance to enable the cam to engage the distal end of the inner sheath when the core is drawn proximally within the inner sheath.

11. The delivery catheter as defined in claim 7 wherein the core extends proximally and distally beyond the proximal and distal ends, respectively, of the outer sheath and is movable independently of each sheath; and
a distal tip is attached to the distal end of the core, the tip having a proximally facing surface adapted to mate smoothly with the distal end of the outer sheath.

12. The delivery catheter as defined in claim 11 wherein the distal end of the outer sheath and the distal tip are radiopaque

13. The delivery catheter as defined in claim 12 wherein the cam is formed integrally with the tip and extends proximally a distance to enable it to engage against the distal end of the inner sheath when the core is drawn proximally within the inner sheath.

14. The delivery catheter as defined in claim 3 further comprising:
the distal region of the inner sheath having a longitudinal groove extending along its outer surface, the groove extending proximally from the socket to the proximal end of the distal region, the groove defining a receptive recess for the strip when in its latched position.

15. The delivery catheter as defined in claim 14 further comprising:
the distal region of the inner sheath having a U-shaped groove extending along its outer surface, the U-shaped groove being defined by two legs joined at an apex, wherein the apex intersects the longitudinal groove at a location immediately proximal to the socket, the legs of the U-shaped groove continuing distally to the end of the distal region of the inner sheath, the U-shaped groove being receptive to the proximal end of the loop of the medical device to maintain the position of the medical device relative to the inner sheath while the strip is in its latched position.

16. An assembly of an implantable expandable medical device and delivery catheter for delivering and deploying the device comprising:
the medical device having a loop at its proximal end;
the catheter having proximal and distal ends and comprising:
an elongate outer sheath having a distal opening, the expandable medical device being contained in the distal portion of the outer sheath in a radially contracted configuration;
an elongate inner sheath extending through the outer sheath, the outer sheath being movable longitudinally over the inner sheath between an extended position in which the distal end of the outer sheath extends distally beyond the distal end of the inner sheath and a retracted position in which the distal end of the outer sheath is disposed proximally of the distal end of the inner sheath, the medical device being contained in the outer sheath when the outer sheath is in its extended position;
a retaining mechanism mounted to the distal region of the inner sheath proximally of the medical device, the retaining mechanism having a latching element that is movable between a latched position in which it securely engages the loop of the device at a single location and an unlatched position in which the loop is released from the latching element; and
a control member disposed within the inner sheath and operatively associated with the latching element to operate the latching element between its latched and unlatched positions.

17. The assembly as defined in claim 16 wherein the retaining mechanism has a follower extending radially into the inner sheath, the control member being longitudinally movable within the inner sheath and engageable with the follower to move the latching element from its latched to its unlatched position.

18. The delivery catheter as defined in claim 17 wherein the control member comprises an elongate core extending through and movable longitudinally through the inner sheath, and a cam mounted to and movable with the core, the core being movable distally of an initial position distal of the follower and proximally of the initial position to an extent to enable the cam to engage the follower to move the latching element to its unlatched position and release the medical device.

19. A method for endovascular placement of an expandable medical device comprising:
providing a delivery catheter containing the medical device in readiness for delivery and deployment, the medical device having proximal and distal ends and a loop at its proximal end, the catheter comprising:
an elongate outer sheath having a distal opening, the medical device being contained in a distal portion of the outer sheath in a radially contracted configuration;
an elongate inner sheath extending through the outer sheath, the outer sheath being movable longitudinally over the inner sheath between an extended position in which the distal end of the outer sheath extends distally beyond the distal end of the inner sheath and a retracted position in which the distal end of the outer sheath is disposed proximally of the distal end of the inner sheath;
a retaining mechanism mounted to the distal region of the inner sheath, the retaining mechanism having a latching element that is movable between a latched position in which it securely engages the loop of the device at a single location and an unlatched position in which the loop is released from the latching element; and
a control member disposed within the inner sheath and operatively associated with the retaining mechanism to operate the latching element between its latched and unlatched positions;
advancing the catheter and medical device intravascularly to a deployment site;
while maintaining the inner sheath in a fixed position, retracting the outer sheath in a proximal direction while maintaining the retaining mechanism in its latched position to maintain the position of the medical device;
observing the position and orientation of the medical device as the outer sheath is withdrawn; and
if the position and orientation of the device is determined satisfactory, operating the control member to move the latching element to its unlatched position to release the device; or
if the position and orientation of the device is determined to be unsatisfactory, maintaining the latching element in its latched position and advancing the outer sheath distally to recapture the device in the outer sheath; and
repositioning the catheter and again operating the catheter to repeat the deployment or removing the catheter and medical device from the patient.
